(19) United States
(12) Patent Application Publication
(10) Pub. No.: US 2021/0121408 A1
(43) Pub. Date: Apr. 29, 2021

(54) Methods of Administering Cannabinoid to People Diagnosed With HIV

(71) Applicants: Catherine Jacobson, San Francisco, CA (US); Philippe Gabriel Lucas, Victoria BC (CA)

(72) Inventors: Catherine Jacobson, San Francisco, CA (US); Philippe Gabriel Lucas, Victoria BC (CA)

(21) Appl. No.: 17/075,656

(22) Filed: Oct. 20, 2020

Related U.S. Application Data
(60) Provisional application No. 62/926,169, filed on Oct. 25, 2019.

(51) Int. Cl. A61K 9/48; A61K 31/05; A61K 31/352; A61K 9/00

(52) U.S. Cl. CPC A61K 9/48; A61K 9/0053; A61K 31/352; A61K 31/05

(57) ABSTRACT

Improved treatments of inflammation caused by HIV using cannabinoids. An exemplary treatment according to the present invention includes administration of an oral solution having a THC+CBD formulation in a 1:1 ratio.

### BACKGROUND OF THE INVENTION

HIV, or human immunodeficiency virus, attacks the immune system's CD4 cells (also known as “T cells”), gradually destroying them and making the body less effective at combating diseases and infections. Untreated, HIV can progress to acquired immunodeficiency syndrome (AIDS), when the immune system is considerably damaged. With AIDS, even diseases and infections that are easily combated by healthy individuals pose a threat that can be fatal. There is currently no effective cure for HIV, so once its acquired, the focus of treatment is on controlling the virus and preventing it from progressing to AIDS.

HIV is a chronic infection. Even patients with an undetectable viral load make new virus. This may contribute to continuing inflammation. Antiretroviral therapy (ART) may reduce the amount of virus (or viral load) in the blood and body fluids, thereby reducing the number of infections that may occur. ART is usually taken as a combination of 3 or more drugs to have the greatest chance of lowering the amount of HIV. ART medications reduce inflammation, but not to normal levels. As HIV weakens the immune system, over time, old infections may return. Inflammation may also be linked to cancer, heart disease, liver and kidney failure, and dementia.

While ART has been shown to effectively curtail HIV from progressing to AIDS, the symptoms and side effects of the drug therapy are considerable. Nausea, vomiting, diarrhea, heart disease, weakened bones, muscle tissue breakdown, and neuropathic pain are commonly reported during HIV treatment regimens. Weight loss due to nausea and a loss of appetite compounds weaknesses in the immune system.

Cannabis may help make the adverse effects more manageable. Daily and chronic neuropathic pain related to HIV may be significantly lowered by regular cannabis consumption. Cannabis also may boost appetite and daily functioning, helping to combat weight loss and muscle breakdown.

Cannabinoids are medicinal agents derived from the cannabis plant (“marijuana”). The two main active components in cannabis are delta-9-tetrahydrocannabinol (THC) and cannabidiol (CBD). THC and CBD are generally thought to exert their actions via the endocannabinoid system. The primary cannabinoid receptors include CB1, found primarily in the brain and in some peripheral tissues, and CB2, found primarily in peripheral tissues.

The molecular formula of both CBD and THC is C₂₁H₃₀O₂. The molecular mass of CBD is 314.46. The partition coefficient of CBD is 7.03. The melting point of CBD is 66-67° C. The boiling point of CBD is 188.5° C. The optical rotation of CBD is −128±5 in Methanol. The hygroscopicity of CBD is <3.0. The UV absorption of CBD is 281.0±1.0 nm. FIG. 1 depicts the structural formula of CBD. The molecular mass of THC is 314.47. The melting point of THC is 200-207° C. The boiling point of THC is 104° C. The optical rotation of THC is −160. The UV Absorption of THC is 280-315 nm. FIG. 2 depicts the structural formula of THC. CBD and THC are insoluble in water, but soluble in ethanol, acetone, chloroform and light petroleum.

### SUMMARY OF THE INVENTION

It is an object of the present invention to provide improved treatments of inflammation caused by HIV using cannabinoids.

An exemplary treatment according to the present invention includes administration of an oral solution having a THC+CBD formulation in a 1:1 ratio.

Numerous variations may be practiced in the preferred embodiment.

### BRIEF DESCRIPTION OF THE DRAWINGS

A further understanding of the invention can be obtained by reference to embodiments set forth in the illustrations of the accompanying drawings. Although the illustrated embodiments are merely examples for carrying out the invention, both the organization and method of operation of the invention, in general, together with further objectives and advantages thereof, may be more easily understood by reference to the drawings and the following description. Like reference numbers generally refer to like features (e.g., functionally similar and/or structurally similar elements).

The drawings are not intended to limit the scope of this invention, which is set forth with particularity in the claims as appended hereto or as subsequently amended, but merely to clarify and exemplify the invention.

FIG. 1 depicts the chemical structure of CBD;

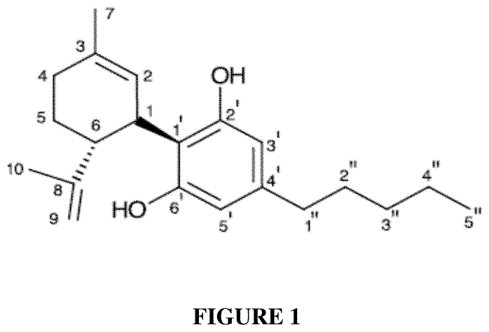

FIG. 2 depicts the chemical structure of THC;

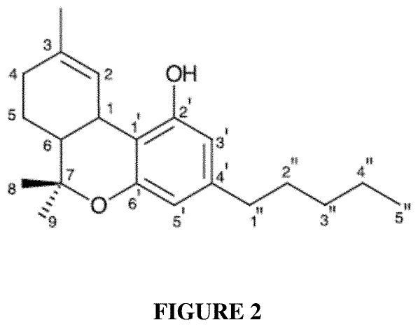

FIG. 3 depicts a table of an exemplary titration schedule;

FIG. 4 depicts a table of an exemplary titration schedule;

FIG. 5 depicts a flow diagram of an exemplary manufacture process in accordance with the present invention;

FIG. 6 depicts an exemplary table of procedures in accordance with the present invention;

FIG. 7A depicts a table of mean pharmacokinetic parameters of THC;

FIG. 7B depicts a table of mean pharmacokinetic parameters of 11-OH-THC;

FIG. 7C depicts a table of mean pharmacokinetic parameters of CBD;

FIG. 7D depicts a table of comparative bioavailability results for plasma THC, 11-OH-THC and CBD under fasting conditions;

FIG. 8A depicts a mean delta-9-tetrahydrocannabinol plasma concentration-time plot following oral administration of a formulation in accordance with the present invention;

FIG. 8B depicts a mean 11-hydroxy-tetrahydrocannabinol (11-OH-THC) plasma concentration-time plot following oral administration of a formulation in accordance with the present invention;

FIG. 8C depicts a mean cannabidiol plasma concentration-time plot following oral administration of a formulation in accordance with the present invention.

### DETAILED DESCRIPTION OF THE INVENTION

The invention may be understood more readily by reference to the following detailed descriptions of embodiments of the invention. However, techniques, systems, and operating structures in accordance with the invention may be embodied in a wide variety of forms and modes, some of which may be quite different from those in the disclosed embodiments. Also, the features and elements disclosed herein may be combined to form various combinations without exclusivity, unless expressly stated otherwise. Consequently, the specific structural and functional details disclosed herein are merely representative. Yet, in that regard, they are deemed to afford the best embodiment for purposes of disclosure and to provide a basis for the claims herein, which define the scope of the invention. It must be noted that, as used in the specification and the appended claims, the singular forms “a”, “an”, and “the” include plural referents unless the context clearly indicates otherwise.

The present invention includes manufacture and administration of oral capsules containing active ingredients CBD and THC. The THC and/or CBD may be extracted from cannabis flower and purified using preparative column chromatography or isolation and crystallization.

The capsules may be made for ingestion and may contain fixed ratios (proportions) of THC to CBD. For example, each capsule may consist of CBD and THC in a 1:1 ratio (e.g., formulated at a strength of 2.5 mg CBD and 2.5 mg THC, per capsule). As another example, each capsule may consist of CBD and THC in a 9:1 ratio (e.g., formulated at a strength of 45 mg CBD and 5 mg THC, per capsule, or 90 mg CBD and 10 mg THC, per capsule).

The capsules may be formulated with standard pharmaceutical excipients. For example, the cannabinoids may be dissolved in Sunflower Lecithin Oil and/or MCT Oil, and may be filled into standard pharmaceutical capsules. Each formulation may be filled into pharmaceutical grade hypromello se capsules.

Example formulation compositions are set forth in the following table.

| | Example 1 | | Example 2 | |
|---|---|---|---|---|
| | Quantity per | | Quantity per | |
| Ingredient | capsule, mg | % | capsule, mg | % |
| CBD (Active) | 2.5 | 0.5 | 45.0 | 9.0 |
| THC (Active) | 2.5 | 0.5 | 5.0 | 1.0 |
| Sunflower Lecithin Oil | 395.0 | 79.0 | 350.0 | 70.0 |
| (Solubilizer) | | | | |
| MCT Oil (Solubilizer) | 100.0 | 20.0 | 100.0 | 20.0 |
| Total per Capsule | 500.0 | 100 | 500.0 | 100 |

Example manufacturing formulas are set forth in the following table.

| | Example 1 | | Example 2 | |
|---|---|---|---|---|
| Batch Size | 7,500 | g | 2,500 | g |
| (number of dosage units) | 15,000 | capsules | 5,000 | capsules |
| Component and Quality | Quantity per batch, g | | Quantity per batch, g | |
| Standard | | | | |
| CBD (Active) | 5.0 | | 90.0 | |
| THC (Active) | 5.0 | | 10.0 | |
| Sunflower Lecithin Oil | 790.0 | | 700.0 | |
| (Solubilizer) | | | | |
| MCT Oil (Solubilizer) | 200.0 | | 200.0 | |
| Total | 1,000 | g | 1.000 | g |

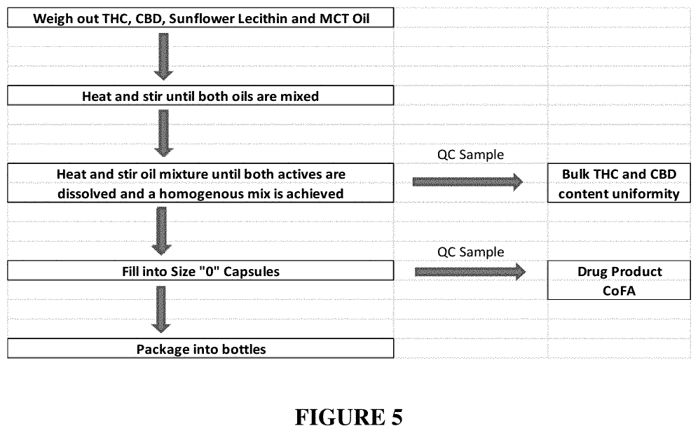

A flow diagram of an exemplary manufacture process is shown in FIG. 5. Both actives, Sunflower Lecithin and MCT Oil may be heated until free flowing (75-85° C.). The Sunflower Lecithin may be mechanically stirred and MCT Oil may be added. The solution may be mechanically stirred at an elevated temperature (75-85° C.) to form a homogenous oil solution. The oil mixture may be mechanically stirred and CBD may be added. The solution may be mechanically stirred at an elevated temperature (75-85° C.) to form a homogenous solution. Next, THC may be added and the solution may be mechanically mixed at an elevated temperature (75-85° C.) to form a homogenous solution. The mixture may then be filled into capsules and the mixture may be maintained fluid at an elevated temperature (75-85° C.).

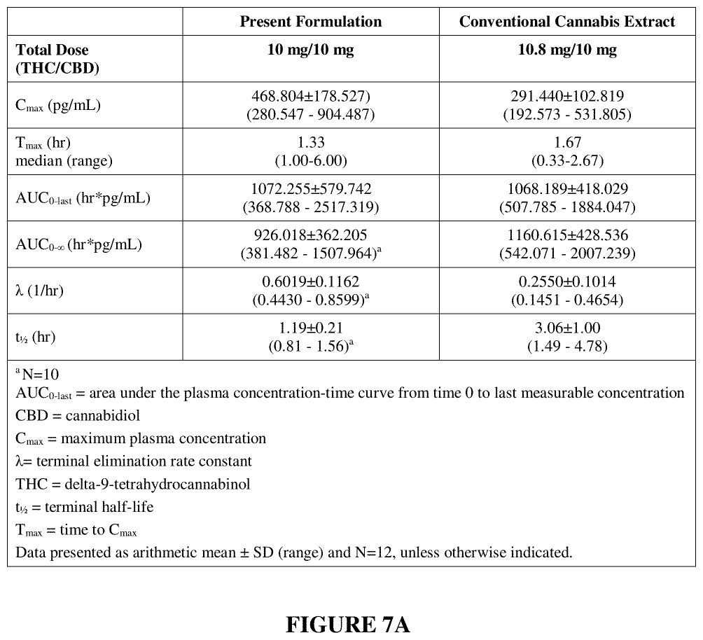

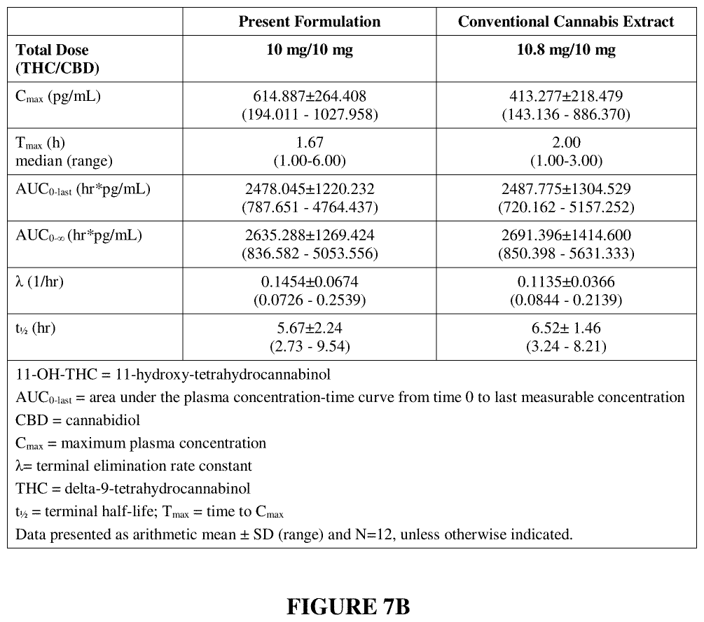

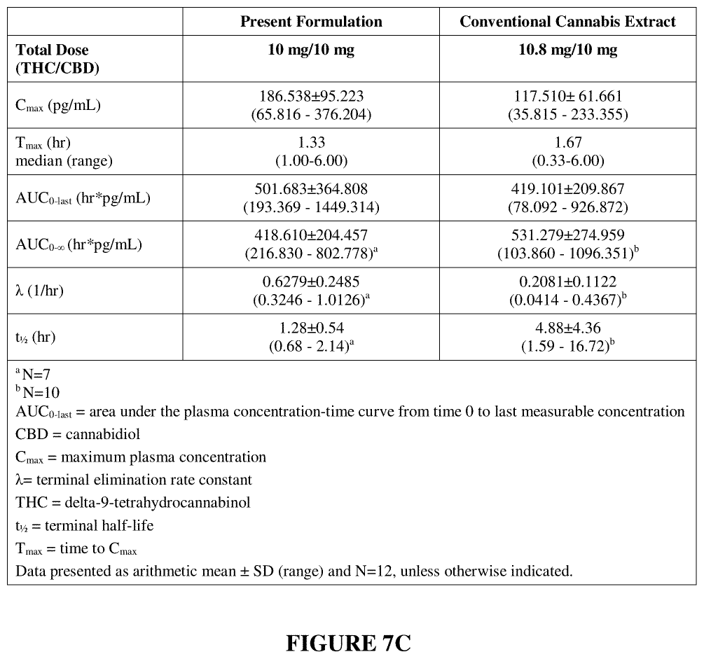

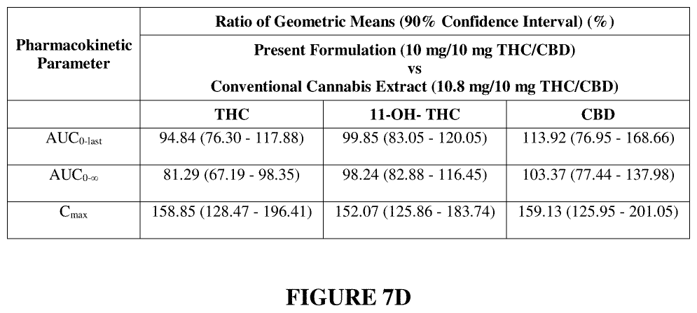

Referring to FIGS. 7A-7C, an open-label, single-center pharmacokinetic study was conducted with twelve healthy volunteers who were administered study drugs under fasting conditions in a randomized order, 7 days apart. Treatment groups of the study drugs included (a) 1 mL oral solution containing 10 mg/10 mg THC/CBD (total dose: 10 mg THC/10 mg CBD), and (b) conventional cannabis extract 4×100 ?L buccal sprays (total dose: 10.8 mg/10 mg THC/CBD). Volunteers were non-smoking, occasional cannabis users (once a month), male and non-pregnant females, 18-53 years of age, inclusive, with a body mass index (BMI) within 20.0-29.5 kg/m2, inclusive. Subjects were confined in in-patient units from at least 10 hours prior to dosing until at least 24 hours post dose, for a total of at least 34 hours. Blood sampling occurred pre-dose (0 hour), and at 0.33, 0.67, 1, 1.33, 1.67, 2, 2.33, 2.67, 3, 3.5, 4, 4.5, 6, 9, 12 and 24 hours post-dose. FIG. 7A is a table summarizing mean pharmacokinetic parameters of THC. FIG. 7B is a table summarizing mean pharmacokinetic parameters of 11-OH-THC. FIG. 7C is a table summarizing mean pharmacokinetic parameters of CBD. The ratio of geometric means and the corresponding 90% confidence intervals for AUCO-t (area under the plasma concentration-time curve), AUCO-?, and Cmax (maximum plasma concentration) for THC, 11-OH-THC and CBD following administration of a formulation in accordance with the present invention or conventional cannabis extract are presented in the table depicted in FIG. 7D.

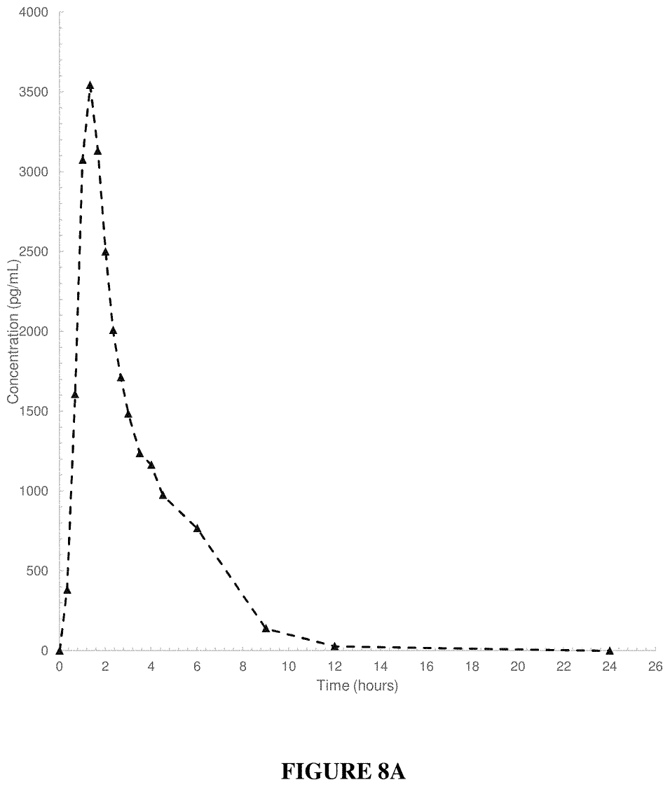

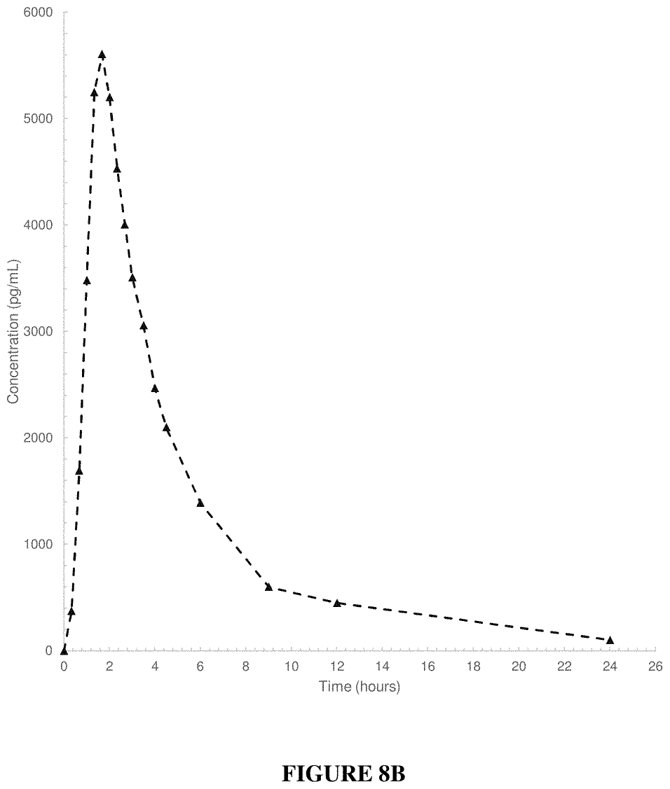

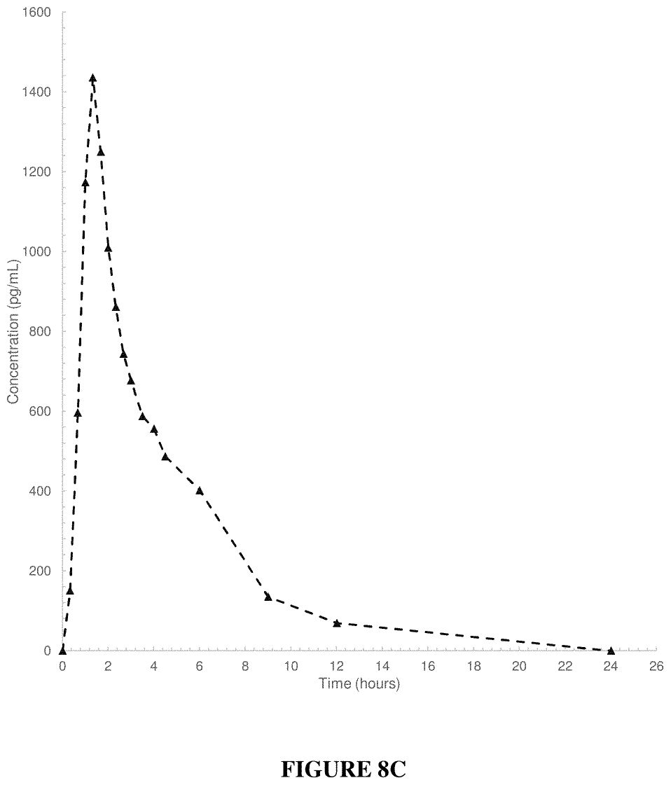

For the study described above, FIGS. 8A-8C depict plasma concentration-time profiles following administration of a formulation in accordance with the present invention. FIG. 8A depicts a mean delta-9-tetrahydrocannabinol (THC) plasma concentration-time plot following oral administration of a formulation in accordance with the present invention. FIG. 8B depicts a mean 11-hydroxy-tetrahydrocannabinol (11-OH-THC) plasma concentration-time plot following oral administration of a formulation in accordance with the present invention. FIG. 8C depicts a mean cannabidiol (CBD) plasma concentration-time plot following oral administration of a formulation in accordance with the present invention.

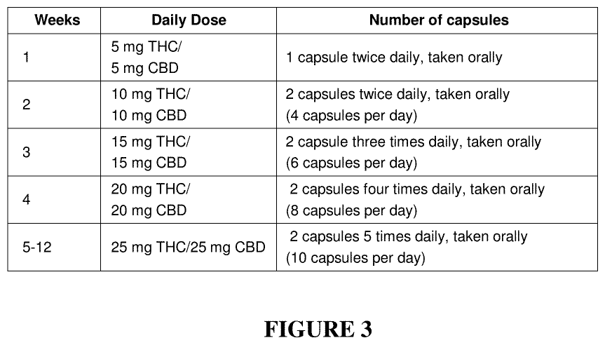

Administration of capsules to persons with HIV according to the present invention may occur, for example, over the course of 12 weeks, a shorter period of time, or a longer period of time. For one method of administration according to the present invention, “low dose” oral capsules (e.g., 2.5 mg THC/2.5 mg CBD capsules) may be administered. Administration may start with 1 capsule twice daily for 1 week (5 mg THC/5 mg CBD), and then increase as tolerated to a maximum of 10 capsules daily by, for example, week 5 (25 mg THC/25 mg CBD total per day). An exemplary titration schedule is shown in FIG. 3. The schedule shown in FIG. 3 includes 12 weeks, but administration may continue beyond that time.

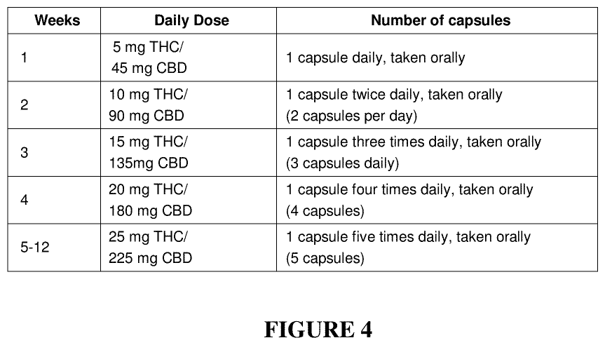

For an alternative method of administration according to the present invention, a higher-dose capsule (e.g., 5 mg THC/45 mg CBD capsules) may be administered once daily for one week, and then increase as tolerated to a maximum of 10 capsules daily by, for example, week five (25 mg THC/225 mg CBD total). An alternative exemplary titration schedule is shown in FIG. 4. The schedule in FIG. 4 includes 12 weeks, but administration may continue beyond that time.

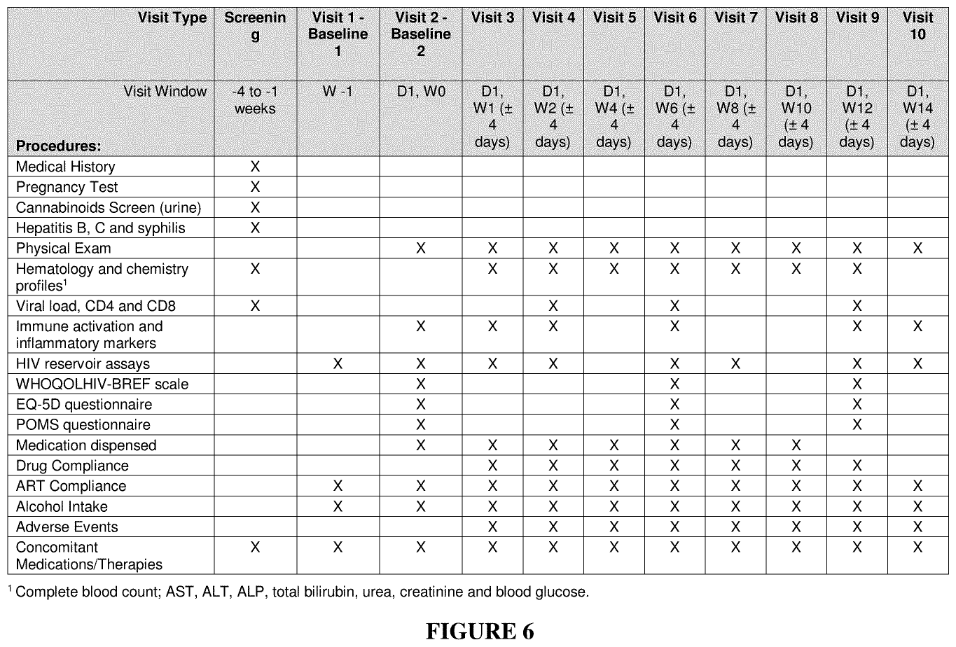

At each week, one or more blood test may be performed to (1) assess the size of the HIV reservoir in the blood; (2) test immune activation and inflammatory markers; (3) conduct hematology and/or chemistry tests; and/or (4) test viral load, CD4 and CD8 cell counts. A sample table of procedures is depicted in FIG. 6.

While the invention has been described in detail with reference to embodiments for the purposes of making a complete disclosure of the invention, such embodiments are merely exemplary and are not intended to be limiting or represent an exhaustive enumeration of all aspects of the invention. It will be apparent to those of ordinary skill in the art that numerous changes may be made in such details, and the invention is capable of being embodied in other forms, without departing from the spirit, essential characteristics, and principles of the invention. Also, the benefits, advantages, solutions to problems, and any elements that may allow or facilitate any benefit, advantage, or solution are not to be construed as critical, required, or essential to the invention. The scope of the invention is to be limited only by the appended claims.

What is claimed is:

1. A method of administering cannabinoids comprising:
oral ingestion of a capsule containing a fixed ratio of CBD to THC, wherein the fixed ratio is 1:1.

2. A method of administering cannabinoids comprising:
oral ingestion of a capsule containing a fixed ratio of CBD to THC, wherein the fixed ratio is 9:1.
